SEQUENCE LISTING (4 sequences)

SEQ ID NO: 1 - DNA, 28 bp, Homo sapiens
ggtgatttgaatgcaaaggaaatccctt

SEQ ID NO: 2 - DNA, 28 bp, Homo sapiens
aagggatttcctttgcattcaaatcacc

SEQ ID NO: 3 - DNA, 28 bp, Homo sapiens
  misc_feature (14)..(14): n is inosine
ggtgatttgaatgnaaaggaaatccctt

SEQ ID NO: 4 - DNA, 28 bp, Homo sapiens
aagggatttcctttccattcaaatcacc

(19) United States
(12) Patent Application Publication
(10) Pub. No.: US 2007/0020639 A1
(43) Pub. Date: Jan. 25, 2007

(54) Isothermal locus specific amplification

(75) Inventor: Michael H. Shapero, Redwood City, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 11/186,120

(22) Filed: Jul. 20, 2005

(51) Int. Cl. C12Q 1/68; C12P 19/34

(52) U.S. Cl. 435/6; 435/91.2

(57) ABSTRACT

Methods are provided for amplifying a template DNA strand using locus-specific primers and enzymes. The method involves denaturing template DNA and then annealing a primer to the single-stranded DNA strand. The primer is then extended using a DNA polymerase. The primer is cleaved downstream of the 3′ end of the inosine base by an endonuclease and subsequently, a first copy of the complementary sequence is displaced. The primer is then extended using a DNA polymerase to form a second extension product. The nicking, displacing, and extending steps are repeated to obtain multiple copies of single stranded DNA complementary to said template DNA sequence.

### FIELD OF THE INVENTION

The methods of the invention relate generally to amplification of a template DNA sample and analysis of the amplified sample.

### BACKGROUND OF THE INVENTION

The past years have seen a dynamic change in the ability of science to comprehend vast amounts of data. Pioneering technologies such as nucleic acid arrays allow scientists to delve into the world of genetics in far greater detail than ever before. Exploration of genomic DNA has long been a dream of the scientific community. Held within the complex structures of genomic DNA lies the potential to identify, diagnose, or treat diseases like cancer, Alzheimer disease or alcoholism.

New techniques such as multiple strand displacement (mda) amplification based on highly processive enzymes have allowed new types of experiments to be conducted when only limiting amounts of genomic DNA samples are available. However, there are applications where it would be beneficial to amplify a certain segment of the genome rather than amplifying the entire genome. This invention discloses a method using locus-specific primers, DNA polymerases, and endonucleases for long-range amplification.

### SUMMARY OF THE INVENTION

A method for obtaining multiple copies of a template DNA strand is disclosed. The method generally includes annealing one or more primers to the template DNA strand, nicking the extension product and extending from the nick. The nicking and extending steps may be repeated multiple times. In some aspects the primer includes a site that serves as a recognition site for a nicking endonuclease. In a preferred aspect, the primer includes an inosine base which is recognized by an Endonuclease V enzyme, which cleaves 2-3 bases 3′ of the inosine. In another aspect, the primer contains a site that is either an abasic site or can be modified to create an abasic site. In a preferred aspect, the primer contains a uracil base and the uracil base is converted to an abasic site by, for example, uracil DNA glycosidase treatment.

In one aspect, a primer containing an inosine is hybridized to a target to form a primer-target complex. The 3′ end of the primer is extended in the presence of DNA polymerase to generate an extended primer that comprises a primer portion and a copy of the template sequence. A nick is generated in the extended primer using an endonuclease that nicks 3′ of the inosine base. The primer region that is 5′ of the nick is extended in the presence of the DNA polymerase, thereby displacing the portion of the extended primer that is 3′ of the nick. The nicking and extension steps are repeated at least once to obtain multiple copies of the template strand.

In a preferred embodiment, the template DNA is genomic DNA. In another preferred embodiment, the primer is a locus specific primer. In a preferred embodiment, the nicking does not remove the inosine base. In a preferred embodiment, the nicking occurs about 2-3 nucleotides downstream of the 3′ end of the inosine base. In another aspect, the primer is 15-200 bases in length, more preferable the primer is 15-100 bases in length, and most preferable the primer is 15-50 bases in length. The DNA polymerase may be, for example, a Klenow fragment (exo-minus) of DNA Polymerase I, Bst DNA polymerase, or phi29 DNA polymerase. Preferably, the polymerase is active between 30° C. and 80° C. In another aspect, the endonuclease is an endonuclease (Endo) V, for example, E. coli endonuclease V or may be thermal stable. In another aspect, Endo V is active between 30° C. and 60° C. In another aspect, a denaturing step may occur before the annealing step. In another aspect, the DNA polymerase reaction and endonuclease reaction are performed in the same buffer, for example, a buffer that contains 20 mM Tris-acetate, 50 mM potassium acetate, 10 mM magnesium acetate and 1 mM DTT, pH 7.9 at 25° C. In another embodiment, the extending, displacing, and nicking steps are performed simultaneously in a single reaction. In another aspect, the extending, displacing, and nicking steps are performed under isothermal conditions. In another aspect, the step of annealing a primer to the template DNA strand comprises mixing the primer with RecA protein to obtain a RecA coated primer and incubating the RecA coated primer with the template DNA strand in the presence of an ATP analogue.

In another aspect, a method is disclosed for amplifying a template DNA. A primer is annealed to a template DNA. The primer is then extended in the presence of a DNA polymerase and deoxyinosine triphosphate to generate a first extension product comprising inosine. The extension product is incubated with Endo V to generate nicks in the first primer extension product at positions 3′ of the incorporated inosine. The nicks are extended with a strand displacing enzyme to generate second extension products. The nicking and extension steps are repeated at least once to generate amplified template DNA. In a preferred embodiment, the ratio of dITP to dGTP incorporation can be 1:10, 1:100 or 1:1,000.

In another aspect, the extension product contains one or more uracils that are converted to an abasic site. The abasic site is recognized by endonuclease V and the extension product is cleaved a few bases 3′ of the abasic site to generate a free 3′ hydroxyl for extension by a strand displacing primer. Uracil may be incorporated into the primer or may be incorporated into the extension product by including dUTP in the extension reaction.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1A shows a template DNA strand and a primer with an inosine base at the cleavage position near the middle of the sequence. The template strand and primer denatures.

FIG. 1B shows annealing of primer to the template DNA. Endonuclease V nicks the 3′ end of the inosine base of the primer. The nick is recognized by the Bst DNA polymerase and the polymerase extends the 3′ end of the primer. The optimal temperature for the annealing and extension steps is at 50° C.

FIG. 1C shows a displaced first copy of a complementary sequence of template DNA. The figure also shows the primer being extended by Bst DNA Polymerase and then the primer is subsequently nicked by Endonuclease V about 2-3 nucleotides downstream of the 3′ side of the inosine base to form a new extension product.

FIG. 2 shows sequences of two artificial duplexes that are each created by annealing two independent oligonucleotides. The first duplex contains no inosine base and consequently, there is no Endo V cleavage. The second duplex contains an inosine base in the top strand and the arrows show Endo V cleavage sites.

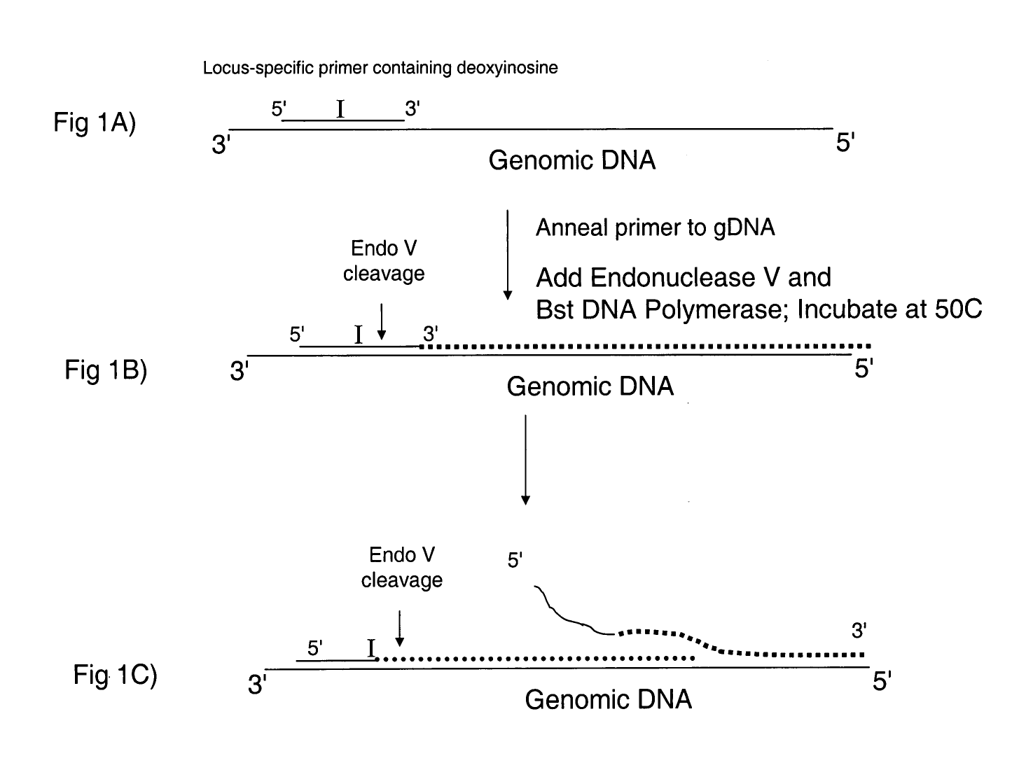

### DETAILED DESCRIPTION OF THE INVENTION

a) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603 each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832, 5,631,734, 5,834,758, 5,936,324, 5,981,956, 6,025,601, 6,141,096, 6,185,030, 6,201,639, 6,218,803, and 6,225,625, in U.S. Ser. No. 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. No. 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/063,559 (United States Publication No. US20020183936), 60/349,546, 60/376,003, 60/394,574 and 60/403,381.

b) Definitions

“Adaptor sequences” or “adaptors” are generally oligonucleotides of at least 5, 10, or 15 bases and preferably no more than 50 or 60 bases in length; however, they may be even longer, up to 100 or 200 bases. Adaptor sequences may be synthesized using any methods known to those of skill in the art. For the purposes of this invention they may, as options, comprise primer binding sites, recognition sites for endonucleases, common sequences and promoters. The adaptor may be entirely or substantially double stranded or entirely single stranded. A double stranded adaptor may comprise two oligonucleotides that are at least partially complementary. The adaptor may be phosphorylated or unphosphorylated on one or both strands.

Adaptors may be more efficiently ligated to fragments if they comprise a substantially double stranded region and a short single stranded region which is complementary to the single stranded region created by digestion with a restriction enzyme. For example, when DNA is digested with the restriction enzyme EcoRI the resulting double stranded fragments are flanked at either end by the single stranded overhang 5′-AATT-3′, an adaptor that carries a single stranded overhang 5′-AATT-3′ will hybridize to the fragment through complementarity between the overhanging regions. This “sticky end” hybridization of the adaptor to the fragment may facilitate ligation of the adaptor to the fragment but blunt ended ligation is also possible. Blunt ends can be converted to sticky ends using the exonuclease activity of the Klenow fragment. For example when DNA is digested with PvuII the blunt ends can be converted to a two base pair overhang by incubating the fragments with Klenow in the presence of dTTP and dCTP. Overhangs may also be converted to blunt ends by filling in an overhang or removing an overhang.

Methods of ligation will be known to those of skill in the art and are described, for example in Sambrook et al. (2001) and the New England BioLabs catalog both of which are incorporated herein by reference for all purposes. Methods include using T4 DNA Ligase which catalyzes the formation of a phosphodiester bond between juxtaposed 5′ phosphate and 3′ hydroxyl termini in duplex DNA or RNA with blunt and sticky ends; Taq DNA Ligase which catalyzes the formation of a phosphodiester bond between juxtaposed 5′ phosphate and 3′ hydroxyl termini of two adjacent oligonucleotides which are hybridized to a complementary target DNA; E. coli DNA ligase which catalyzes the formation of a phosphodiester bond between juxtaposed 5′-phosphate and 3′-hydroxyl termini in duplex DNA containing cohesive ends; and T4 RNA ligase which catalyzes ligation of a 5′ phosphoryl-terminated nucleic acid donor to a 3′ hydroxyl-terminated nucleic acid acceptor through the formation of a 3′->5′ phosphodiester bond, substrates include single-stranded RNA and DNA as well as dinucleoside pyrophosphates; or any other methods described in the art. Fragmented DNA may be treated with one or more enzymes, for example, an endonuclease, prior to ligation of adaptors to one or both ends to facilitate ligation by generating ends that are compatible with ligation.

Adaptors may also incorporate modified nucleotides that modify the properties of the adaptor sequence. For example, phosphorothioate groups may be incorporated in one of the adaptor strands. A phosphorothioate group is a modified phosphate group with one of the oxygen atoms replaced by a sulfur atom. In a phosphorothioated oligo (often called an “S-Oligo”), some or all of the internucleotide phosphate groups are replaced by phosphorothioate groups. The modified backbone of an S-Oligo is resistant to the action of most exonucleases and endonucleases. Phosphorothioates may be incorporated between all residues of an adaptor strand, or at specified locations within a sequence. A useful option is to sulfurize only the last few residues at each end of the oligo. This results in an oligo that is resistant to exonucleases, but has a natural DNA center.

The term “array” as used herein refers to an intentionally created collection of molecules which can be prepared either synthetically or biosynthetically. The molecules in the array can be identical or different from each other. The array can assume a variety of formats, for example, libraries of soluble molecules; libraries of compounds tethered to resin beads, silica chips, or other solid supports.

The term “array plate” as used herein refers to a body having a plurality of arrays in which each microarray is separated by a physical barrier resistant to the passage of liquids and forming an area or space, referred to as a well, capable of containing liquids in contact with the probe array.

The term “biomonomer” as used herein refers to a single unit of biopolymer, which can be linked with the same or other biomonomers to form a biopolymer (for example, a single amino acid or nucleotide with two linking groups one or both of which may have removable protecting groups) or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

The term “biopolymer” or sometimes referred by “biological polymer” as used herein is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above.

The term “combinatorial synthesis strategy” as used herein refers to a combinatorial synthesis strategy is an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “epigenetic” as used herein refers to factors other than the primary sequence of the genome that affect the development or function of an organism, they can affect the phenotype of an organism without changing the genotype. Epigenetic factors include modifications in gene expression that are controlled by heritable but potentially reversible changes in DNA methylation and chromatin structure. Methylation patterns are known to correlate with gene expression and in general highly methylated sequences are poorly expressed.

The term “genome” as used herein is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” Hybridizations are usually performed under stringent conditions, for example, at a salt concentration of no more than about 1 M and a temperature of at least 25° C. For example, conditions of 5×SSPE (750 mM NaCl, 50 mM NaPhosphate, 5 mM EDTA, pH 7.4) and a temperature of 25° C.-30° C. are suitable for allele-specific probe hybridizations or conditions of 100 mM MES, 1 M [Na⁺], 20 mM EDTA, 0.01% Tween-20 and a temperature of 30° C.-50° C., preferably at about 45° C.-50° C. Hybridizations may be performed in the presence of agents such as herring sperm DNA at about 0.1 mg/ml, acetylated BSA at about 0.5 mg/ml. As other factors may affect the stringency of hybridization, including base composition and length of the complementary strands, presence of organic solvents and extent of base mismatching, the combination of parameters is more important than the absolute measure of any one alone. Hybridization conditions suitable for microarrays are described in the Gene Expression Technical Manual, 2004 and the GeneChip Mapping Assay Manual, 2004, available at Affymetrix.com.

The term “hybridization probes” as used herein are oligonucleotides capable of binding in a base-specific manner to a complementary strand of nucleic acid. Such probes include peptide nucleic acids, as described in Nielsen et al., Science 254, 1497-1500 (1991), LNAs, as described in Koshkin et al. Tetrahedron 54:3607-3630, 1998, and U.S. Pat. No. 6,268,490 and other nucleic acid analogs and nucleic acid mimetics.

The term “isolated nucleic acid” as used herein mean an object species invention that is the predominant species present (i.e., on a molar basis it is more abundant than any other individual species in the composition). Preferably, an isolated nucleic acid comprises at least about 50%, 80% or 90% (on a molar basis) of all macromolecular species present. Most preferably, the object species is purified to essential homogeneity (contaminant species cannot be detected in the composition by conventional detection methods).

The term “label” as used herein refers to a luminescent label, a light scattering label or a radioactive label. Fluorescent labels include, inter alia, the commercially available fluorescein phosphoramidites such as Fluoreprime (Pharmacia), Fluoredite (Millipore) and FAM (ABI). See U.S. Pat. No. 6,287,778.

The term “ligand” as used herein refers to a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (for example, opiates, steroids, etc.), hormone receptors, peptides, enzymes, enzyme substrates, substrate analogs, transition state analogs, cofactors, drugs, proteins, and antibodies.

Linkage disequilibrium or allelic association means the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles.

The term “mixed population” or sometimes refer by “complex population” as used herein refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but includes other undesirable populations. For example, a complex population of nucleic acids may be a sample which has been enriched for desired messenger RNA (mRNA) sequences but still includes some undesired ribosomal RNA sequences (rRNA).

The term “mRNA” or sometimes refer by “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “nucleic acid library” as used herein refers to an intentionally created collection of nucleic acids which can be prepared either synthetically or biosynthetically and screened for biological activity in a variety of different formats (for example, libraries of soluble molecules; and libraries of oligos tethered to beads, chips, or other solid supports). Additionally, the term “array” is meant to include those libraries of nucleic acids which can be prepared by spotting nucleic acids of essentially any length (for example, from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 5%, 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

The term “receptor” as used herein refers to a molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptor is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term target is used herein, no difference in meaning is intended. A “Probe Target Pair” is formed when two macromolecules have combined through molecular recognition to form a complex.

The term “wafer” as used herein refers to a substrate having surface to which a plurality of arrays are bound. In a preferred embodiment, the arrays are synthesized on the surface of the substrate to create multiple arrays that are physically separate. In one preferred embodiment of a wafer, the arrays are physically separated by a distance of at least about 0.1, 0.25, 0.5, 1 or 1.5 millimeters. The arrays that are on the wafer may be identical, each one may be different, or there may be some combination thereof. Particularly preferred wafers are about 8″×8″ and are made using the photolithographic process.

The term “isothermal amplification” refers to an amplification reaction that is conducted at a substantially constant temperature. The isothermal portion of the reaction may be proceeded by or followed by one or more steps at a variable temperature, for example, a first denaturation step and a final heat inactivation step or cooling step. It will be understood that this definition by no means excludes certain, preferably small, variations in temperature but is rather used to differentiate the isothermal amplification techniques from other amplification techniques known in the art that basically rely on “cycling temperatures” in order to generate the amplified products. Isothermal amplification, varies from, for example PCR, in that PCR amplification relies on cycles of denaturation by heating followed by primer hybridization and polymerization at a lower temperature.

The term “Strand Displacement Amplification” (SDA) is an isothermal in vitro method for amplification of nucleic acid. In general, SDA methods initiate synthesis of a copy of a nucleic acid at a free 3′ OH that may be provided, for example, by a primer that is hybridized to the template. The DNA polymerase extends from the free 3′ OH and in so doing, displaces the strand that is hybridized to the template leaving a newly synthesized strand in its place. Subsequent rounds of amplification can be primed by a new primer that hybridizes 5′ of the original primer or by introduction of a nick in the original primer. Repeated nicking and extension with continuous displacement of new DNA strands results in exponential amplification of the original template. Methods of SDA have been previously disclosed, including use of nicking by a restriction enzyme where the template strand is resistant to cleavage as a result of hemimethylation. Another method of performing SDA involves the use of “nicking” restriction enzymes that are modified to cleave only one strand at the enzymes recognition site. A number of nicking restriction enzymes are commercially available from New England Biolabs and other commercial vendors.

Polymerases useful for SDA generally will initiate 5′ to 3′ polymerization at a nick site, will have strand displacing activity, and preferably will lack substantial 5′ to 3′ exonuclease activity. Enzymes that may be used include, for example, the Klenow fragment of DNA polymerase I, Bst polymerase large fragment, Phi29, and others. DNA Polymerase I Large (Klenow) Fragment consists of a single polypeptide chain (68 kDa) that lacks the 5′ to 3′ exonuclease activity of intact E. coli DNA polymerase I. However, DNA Polymerase I Large (Klenow) Fragment retains its 5′ to 3′ polymerase, 3′ to 5′ exonuclease and strand displacement activities. The Klenow fragment has been used for SDA. For methods of using Klenow for SDA see, for example, U.S. Pat. Nos. 6,379,888; 6,054,279; 5,919,630; 5,856,145; 5,846,726; 5,800,989; 5,766,852; 5,744,311; 5,736,365; 5,712,124; 5,702,926; 5,648,211; 5,641,633; 5,624,825; 5,593,867; 5,561,044; 5,550,025; 5,547,861; 5,536,649; 5,470,723; 5,455,166; 5,422,252; 5,270,184, the disclosures of which are incorporated herein by reference. Examples of other enzymes that may be used include: exo minus Vent (NEB), exo minus Deep Vent (NEB), Bst (BioRad), exo minus Pfu (Stratagene), Pfx (Invitrogen), 9° Nₘ™ (NEB), and other thermostable polymerases.

Phi29 is a DNA polymerase from Bacillus subtilis that is capable of extending a primer over a very long range, for example, more than 10 Kb and up to about 70 Kb. This enzyme catalyzes a highly processive DNA synthesis coupled to strand displacement and possesses an inherent 3′ to 5′ exonuclease activity, acting on both double and single stranded DNA. Variants of phi29 enzymes may be used, for example, an exonuclease minus variant may be used. Phi29 DNA Polymerase optimal temperature range is between about 30° C. to 37° C., but the enzyme will also function at higher temperatures and may be inactivated by incubation at about 65° C. for about 10 minutes. Phi29 DNA polymerase and Tma Endonuclease V (available from Fermentas Life Sciences) are active under compatible buffer conditions. Phi29 is 90% active in NEBuffer 4 (20 mM Tris-acetate, 50 mM potassium acetate, 10 mM magnesium acetate and 1 mM DTT, pH 7.9 at 25° C.) and is also active in NEBuffer 1 (10 mM Bis-Tris-Propane-HCl, 10 mM magnesium chloride and 1 mM DTT, pH 7.0 at 25° C.), NEBuffer 2 (50 mM sodium chloride, 10 mM Tris-HCl, 10 mM magnesium chloride and 1 mM DTT, pH 7.9 at 25° C.), NEBuffer 3 (100 mM NaCl, 50 mM Tris HCl, 10 mM magnesium chloride and 1 mM DTT, pH 7.9 at 25° C.). For additional information on phi29, see U.S. Pat. Nos. 5,100,050, 5,198,543 and 5,576,204.

Bst DNA polymerase originates from Bacillus stearothermophilus and has a 5′ to 3′ polymerase activity, but lacks a 5′ to 3′ exonuclease activity. This polymerase is known to have strand displacing activity. The enzyme is available from, for example, New England Biolabs. Bst is active at high temperatures and the reaction may be incubated optimally at about 65° C. but also retains 30%-45% of its activity at 50° C. Its active range is between 37° C.-80° C. The enzyme tolerates reaction conditions of 70° C. and below and can be heat inactivated by incubation at 80° C. for 10 minutes. Bst DNA polymerase is active in the NEBuffer 4 (20 mM Tris-acetate, 50 mM potassium acetate, 10 mM magnesium acetate and 1 mM DTT, pH 7.9 at 25° C.) as well as NEBuffer 1 (10 mM Bis-Tris-Propane-HCl, 10 mM magnesium chloride and 1 mM DTT, pH 7.0 at 25° C.), NEBuffer 2 (50 mM sodium chloride, 10 mM Tris-HCl, 10 mM magnesium chloride and 1 mM DTT, pH 7.9 at 25° C.), and NEBuffer 3 (100 mM NaCl, 50 mM Tris HCl, 10 mM magnesium chloride and 1 mM DTT, pH 7.9 at 25° C.). Bst DNA polymerase could be used in conjunction with E. coli Endonuclease V (available from New England Biolabs). For additional information see Mead, D. A. et al. (1991) BioTechniques, p.p. 76-87, McClary, J. et al. (1991) J. DNA Sequencing and Mapping, p.p. 173-180 and Hugh, G. and Griffin, M. (1994) PCR Technology, p.p. 228-229.

The term “endonuclease” refers to an enzyme that cleaves a nucleic acid (DNA or RNA) at internal sites in a nucleotide base sequence. Cleavage may be at a specific recognition sequence, at sites of modification or randomly. Specifically, their biochemical activity is the hydrolysis of the phosphodiester backbone at sites in a DNA sequence. Examples of endonucleases include Endonuclease V (Endo V) also called deoxyinosine 3′ endonuclease, which recognizes DNA containing deoxyinosines (paired or not). Endonuclease V cleaves the second and third phosphodiester bonds 3′ to the mismatch of deoxyinosine with a 95% efficiency for the second bond and a 5% efficiency for the third bond, leaving a nick with 3′ hydroxyl and 5′ phosphate. Endo V, to a lesser, degree, also recognizes DNA containing abasic sites and also DNA containing urea residues, base mismatches, insertion/deletion mismatches, hairpin or unpaired loops, flaps and pseudo-Y structures. See also, Yao et al., J. Biol. Chem., 271(48): 30672 (1996), Yao et al., J. Biol. Chem., 270(48): 28609 (1995), Yao et al., J. Biol. Chem., 269(50): 31390 (1994), and He et al., Mutat. Res., 459(2):109 (2000). Endo V from E. coli is active at temperatures between about 30 and 50° C. and preferably is incubated at a temperature between about 30° C. to 37° C. Endo V is active in NEBuffer 4 (20 mM Tris-acetate, 50 mM potassium acetate, 10 mM magnesium acetate and 1 mM DTT, pH 7.9 at 25° C.), but is also active in other buffer conditions, for example, 20 mM HEPES-NaOH (pH 7.4), 100 mM KCl, 2 mM MnCl₂ and 0.1 mg/ml BSA. Endo V makes a strand specific nick about 2-3 nucleotides downstream of the 3′ side of inosine base, without removing the inosine base. Endonucleases, including Endo V, may be obtained from manufacturers such as New England Biolabs (NEB) or Fermentas Life Sciences.

The RecA protein is a protein found in E. coli that in the presence of ATP, promotes the strand exchange of single-strand DNA fragments with homologous duplex DNA. RecA is also an ATPase, an enzyme capable of hydrolyzing ATP, when bound to DNA. RecA uses ATP to carry out strand exchange over long sequences and impose direction to the exchange, to bypass short sequence heterogeneities, and to stall replication so DNA lesions can be mended. The reaction has three distinct steps: (i) RecA polymerizes on the single-strand DNA to form a nucleoprotein filament, (ii) the nucleoprotein filament binds the duplex DNA and searches for a homologous region in a process that requires ATP but not hydrolysis, because ATPγS, a noncleavable analogue, can substitute, (iii) RecA catalyzes local denaturation of the duplex and strand exchange with the single-stranded DNA, see also Radding, C. M. (1991) J. Biol. Chem., 266: 5355-5358. Recombinant E.coli RecA is commercially available from, for example, New England Biolabs. The use of a nonhydrolyzable analogue such as ATPγS favors the formation of stable triple stranded complexes. For reaction conditions useful for promoting oligonucleotide binding to a duplex DNA, see Rigas et al. Proc. Natl. Acad. Sci. USA 83:9591-9595 (1986) and Honigberg et al. Proc. Natl. Acad. Sci. USA 83:9586-9590 (1986). RecA is active under a variety of reaction conditions and can be heat inactivated at 65° C. for 20 minutes.

c) Isothermal Locus-Specific Amplification

The invention provides methods and compositions for polynucleotide amplification, as well as applications of the amplification methods. Nucleic acid amplification has extensive applications in gene expression profiling, genetic testing, diagnostics, environmental monitoring, resequencing, forensics, drug discovery, pharmacogenomics and other areas. Nucleic acid samples may be derived, for example, from total nucleic acid from a cell or sample, total RNA, cDNA, genomic DNA or mRNA. Many methods of analysis of nucleic acid employ methods of amplification of the nucleic acid sample prior to analysis. A number of methods for the amplification of nucleic acids have been described, for example, exponential amplification, linked linear amplification, ligation-based amplification, and transcription-based amplification. An example of exponential nucleic acid amplification method is polymerase chain reaction (PCR) which has been disclosed in numerous publications. See, for example, Mullis et al. Cold Spring Harbor Symp. Quant. Biol. 51:263-273 (1986); and U.S. Pat. Nos. 4,582,788 and 4,683,194.

Nucleic acid amplification may be carried out through multiple cycles of incubations at various temperatures, i.e. thermal cycling or PCR, or at a constant temperature (an isothermal process). An example of an isothermal amplification technique involves a single, elevated temperature using a DNA polymerase that contains the 5′ to 3′ polymerase activity but lacks the 5′ to 3′ exonuclease activity. As the new strand of DNA is synthesized from the template strand of DNA, the complementary strand of the DNA target is displaced from the original DNA helix. The use of specific primers that invade the target DNA strand allows for self-sustaining amplification and detection techniques and can detect very low copy targets. Isothermal amplification methods, such as strand displacement amplification (SDA), are disclosed in U.S. Pat. Nos. 5,648,211, 5,824,517, 6,858,413, 6,692,918, 6,686,156, 6,251,639 and 5,744,311 and U.S. Patent Pub. No. 20040115644 and in Walker et al. Proc. Natl. Acad. Sci. U.S.A. 89: 392-396 (1992); Guatelli, J. C. et al. Proc. Natl. Acad. Sci. USA 87:1874-1878 (1990); which are incorporated herein by reference in their entirety.

When a pair of amplification primers is used, each of which hybridizes to one of the two strands of a double stranded target sequence, amplification is exponential. This is because the newly synthesized strands serve as templates for the opposite primer in subsequent rounds of amplification. When a single amplification primer is used, amplification is linear because only one strand serves as a template for primer extension and newly synthesized strands are not used as template. Amplification methods that proceed linearly during the course of the amplification reaction are less likely to introduce bias in the relative levels of different mRNAs than those that proceed exponentially. “Single-primer amplification” protocols have been reported in many patents (see, for example, U.S. Pat. Nos. 5,554,516, 5,716,785, 6,132,997, 6,251,639, and 6,692,918 which are incorporated herein by reference in their entirety).

Nucleic acid amplification techniques may be grouped according to the temperature requirements of the procedure. Certain nucleic acid amplification methods, such as the polymerase chain reaction (PCR.TM.—Saiki et al., Science, 230:1350-1354, 1985), ligase chain reaction (LCR—Wu et al., Genomics, 4:560-569, 1989; Barringer et al., Gene, 89:117-122, 1990; Barany, Proc. Natl. Sci. USA, 88:189-193, 1991), transcription-based amplification (Kwoh et al., Proc. Natl. Acad. Sci., USA, 86:1173-1177, 1989) and restriction amplification (U.S. Pat. No. 5,102,784), require temperature cycling of the reaction between high denaturing temperatures and somewhat lower polymerization temperatures. In contrast, methods such as self-sustained sequence replication (Guatelli et al., Proc. Natl. Acad. Sci. USA, 87:1874-1878, 1990), the Q.beta. replicase system (Lizardi et al., BioTechnology, 6:1197-1202, 1988), and Strand Displacement Amplification (SDA—Walker et al., Proc. Natl. Acad. Sci. USA, 89:392-396, 1992a, Walker et al., Nuc. Acids. Res., 20:1691-1696, 1992b; U.S. Pat. No. 5,455,166) are isothermal reactions that are conducted at a constant temperature, which are typically much lower than the reaction temperatures of temperature cycling amplification methods.

The Strand Displacement Amplification (SDA) reaction initially developed was conducted at a constant temperature between about 37° C. and 42° C. (U.S. Pat. No. 5,455,166). This temperature range was selected because the exo-klenow DNA polymerase and the restriction endonuclease (e.g., HindII) are mesophilic enzymes that are thermolabile (temperature sensitive) at temperatures above this range. The enzymes that drive the amplification are therefore inactivated as the reaction temperature is increased. Isothermal SDA may also be performed at higher temperatures, for example, 50° C. to 70° C. by using enzymes that are thermostable. Thermophilic SDA is described in European Patent Application No. 0 684 315 and employs thermophilic restriction endonucleases that nick the hemimodified restriction endonuclease recognition/cleavage site at high temperature and thermophilic polymerases that extend from the nick and displace the downstream strand in the same temperature range.

Methods for isothermal SDA are disclosed herein. In a preferred embodiment, a primer that includes a modified base that is recognized by an endonuclease is used to primer synthesis. The primer is then cleaved by the endonuclease to generate a nick in the primer 3′ of the modified base. The nick serves as a point of initiation for synthesis of a new strand by a strand displacing enzyme, displacing the previous extension product.

The primer containing the modified base is first hybridized to the template strand; the template may first be denatured to facilitate hybridization of the primer. The annealed primer is then extended with a DNA polymerase that preferably has strand displacing activity. The newly synthesized strand, which includes the primer and the first extension product, is then cleaved by an endonuclease that recognizes the inosine base in the primer. In a preferred aspect, nicking occurs 3′ of the inosine base so that the modified base remains unaffected. The nicking generates a free 3′ OH within the primer that can be extended to generate a second extension product that displaces the first extension product. The nicking, extending and displacing steps are repeated at least once to obtain multiple copies of single-stranded DNA complementary to the template DNA sequence. In a preferred embodiment, the template DNA is genomic DNA. The primer may be, for example, a locus specific primer, a collection of locus specific primers or a collection of random or a collection of degenerate or partially degenerate primers.

The primer is preferably between 15 to 200 bases in length, more preferable 15 to 100 bases in length or most preferable 15 to 50 bases in length. In a preferred embodiment, the DNA polymerase extends the 3′ end of the primer and contains 3′ to 5′ exonuclease activity. DNA polymerases that may be used include, for example, Klenow fragment, Bst polymerase, and phi29 polymerase. In some aspects Bst DNA polymerase is used. Bst DNA polymerase is thermal stable and reactions are preferably incubated at about 65° C., the enzyme is also active at lower temperature, for example, the enzyme retains 30%-45% of its activity at 50° C. In another preferred embodiment, phi29 DNA polymerase is used. Phi29 has an optimal temperature range of about 30° C.-37° C. If an initial denaturation step is being used, the enzymes are preferably added after denaturation. The denaturation step takes place at about 95° C. while the annealing step takes place at about 50° C. Bst DNA polymerase and phi29 DNA polymerase have strong strand displacement activity, so any products generated from the natural 3′ end of the original primer or from a prior nick will be displaced by new products made from the extending nick. In a preferred embodiment, the nicking, extending, and displacing steps are performed simultaneously in a single reaction, preferably under isothermal conditions. In many aspects the strand displacing polymerase and the nicking endonuclease are active under the same reaction conditions and within the same temperature range. Bst DNA Polymerase, and Endo V from E. coli, are active under similar buffer conditions, for example a buffer that consists of 20 mM Tris-acetate, 50 mM potassium acetate, 10 mM magnesium acetate and 1 mM DTT, pH 7.9 at 25° C. Compositions of other buffers that could be used include: 10 mM Bis-Tris-Propane-HCl, 10 mM magnesium chloride and 1 mM DTT, pH 7.0 at 25° C.; 50 mM sodium chloride, 10 mM Tris-HCl, 10 mM magnesium chloride and 1 mM DTT, pH 7.9 at 25°C.; 100 mM NaCl, 50 mM Tris HCl, 10 mM magnesium chloride and 1 mM DTT, pH 7.9 at 25° C. In preferred aspects the polymerase extends the primer more than 10,000 bases, more than 100,000 bases or more than 1,000,000 bases. Ultra long extension may result in the use of a relatively small number of locus specific primers to generate amplification of one or more genomic regions of interest.

In a preferred embodiment, the endonuclease is Endonuclease V (Endo V). Endo V will also cleave 3′ of an abasic site. Endo V is a repair enzyme found in E. coli that recognizes deoxyinosine, a deamination product of deoxyadenosine in DNA. Endo V, often called deoxyinosine 3′ endonuclease, recognizes DNA containing deoxyinosines (paired or not) on double-stranded DNA, single-stranded DNA with deoxyinosines and to a lesser degree other damages in DNA, for example, DNA containing abasic sites (ap) or urea, base mismatches, insertion/deletion mismatches, hairpin or unpaired loops, flaps and pseudo-Y structures. Endo V does not remove the deoxyinsoine or the damaged bases. Endo V cleaves the second and third phosphodiester bonds 3′ to the mismatch of deoxyinosine with a 95% efficiency for the second bond and a 5% efficiency for the third bond, leaving a nick with 3′-hydroxyl and 5′-phosphate. The optimal temperature range of E. coli Endo V is about 30° C. to 37° C. but the enzyme is active between 30° C. to 60° C. Endo V from E. coli is commercially available from, for example, New England Biolabs. Thermal stable Endo V is also commercially available, for example, Tma (Fermentas Life Sciences). The nick is made downstream of the inosine base leaving the inosine base 5′ of the nick, so the process can repeat itself many times. In preferred aspects a thermal stable strand displacing enzyme, for example, Bst DNA Polymerase is paired in a reaction with a thermal stable Endo V, for example, Tma. In another aspect, Phi29 is paired with Endo V. In preferred aspects, the endo V and the polymerase are active under the same buffer and reaction conditions, including temperature.

In another aspect, annealing of the primer to the template strand is facilitated by the use of the RecA protein. The inosine containing primer is coated with one or more Rec A proteins and incubated with the duplex DNA strand in the presence of an ATP analogue to form nucleofilaments. The nucleofilaments, are added to the intact template DNA strand in the presence of an ATP analogue. The primer finds its complementary sequence and then anneals to the template and the extending, nicking, and displacing steps are as disclosed above. In a preferred aspect, an E. coli RecA protein is used. Rec A protein is active in a variety of buffer conditions, for example, 20 mM Tris-HCl (pH 7.5), 10 mM MgCl₂, 1 mM DTT. About 50 μg of RecA may be incubated with about 0.5-5 μg of DNA at 30° C. for about 1-4 or 4-16 hours in a 50 μl reaction volume. For additional reaction conditions for RecA protein see, for example, Koob et al. (1992) R. Wu (Eds.) Methods in Enzymology, 216, pp. 321-329 (1992).

In another embodiment, inosine bases may be incorporated at low levels into the DNA polymerase product. For example, phi29 DNA polymerase can incorporate dITP bases opposite cytosine bases. By titrating in a small amount of dITP with dGTP, the inosine serves as a base along the growing product that can recognized by Endo V. These nicks will serve as new points of initiation for the DNA polymerase. This method should allow polymerization to extend farther from the original primer. The starting ratios of dITP to dGTP may be, for example, 1:10, 1:100, or 1:1,000.

In another aspect, the primer has one or more uracil bases or uracil is incorporated in the extension product. The extension product may be treated with uracil DNA glycosidase to generate an abasic site at a uracil and Endo V may be used to cleave 3′ of the abasic site to generate an extendable nick.

After incubation for a period of time (for example, several hours), the reaction may be passed over a column, for example, a S-400 column, to remove unincorporated primer. The reaction may be diluted, and optionally used in a phi29 reaction with random hexamers to allow for amplification of both strands and to increase the mass of the target prior to processing for array hybridization. Kits for amplification using phi29 and random primers are commercially available, for example, GenomiPhi (Amersham) or REPLI-g (Qiagen). This material may be purified, fragmented with DNase I, and end-labeled with TdT and DLR and hybridized to an array, for example, a SNP genotyping array such as the Mapping 100K array from Affymetrix.

The fragmentation process produces DNA fragments within a certain range of length that can subsequently be labeled. The average size of fragments obtained is at least 10, 20, 30, 40, 50, 60, 70, 80, 100 or 200 nucleotides. Fragmentation of nucleic acids comprises breaking nucleic acid molecules into smaller fragments. Fragmentation of nucleic acid may be desirable to optimize the size of nucleic acid molecules for certain reactions and destroy their three dimensional structure. For example, fragmented nucleic acids may be used for more efficient hybridization of target DNA to nucleic acid probes than non-fragmented DNA. According to a preferred embodiment, before hybridization to a microarray, target nucleic acid should be fragmented to sizes ranging from 50 to 200 bases long to improve target specificity and sensitivity.

Labeling may be performed before or after fragmentation using any suitable methods. The amplified fragments are labeled with a detectable label such as biotin and hybridized to an array of target specific probes, such as those available from Affymetrix under the brand name GENECHIP®. Labeling methods are well known in the art and are discussed in numerous references including those incorporated by reference.

Multiple copies of DNA generated by the disclosed methods are analyzed by hybridization to an array of probes. The nucleic acids generated by the methods may be analyzed by hybridization to nucleic acid arrays. One of skill in the art would appreciate that the amplification products generated by the methods are suitable for use with many methods for analysis of nucleic acids. One of skill in the art will appreciate that an enormous number of array designs are suitable for the practice of this invention. High density arrays may be used for a variety of applications, including, for example, gene expression analysis, genotyping and variant detection. Array based methods for monitoring gene expression are disclosed and discussed in detail in U.S. Pat. Nos. 5,800,992, 5,871,928, 5,925,525, 6,040,138 and PCT Application WO92/10588 (published on Jun. 25, 1992). Suitable arrays are available, for example, from Affymetrix, Inc. (Santa Clara, Calif.).

### EXAMPLES

### Example 1

### Cleavage of Inosine Containing Primers Using Endo V

Five different primers were digested with E. coli Endo V. The first primer had no inosine in the sequence and did not digest with Endo V. The remaining 4 primers each contained inosine and were digested with the Endo V. Reactions were carried out in 1× NEBuffer 4 at 37° C. for 1 hour. The results showed that no non-specific activity was detected from Endo V when a single stranded oligonucleotide template was used. Products were run on an 8M Urea gel, 15% acrylamide and stained with Sybr Gold stain.

### Example 2

### Endo V Activity in phi29 Reaction

In another example, E. coli Endo V was tested for activity in 1× phi29 DNA polymerase reaction buffer to determine if phi29 and Endo V are active in the same buffer. The same level of cleavage was observed when either NEBuffer 4 was used or when phi29 buffer was used, demonstrating that Endo V is active in phi29 reaction buffer. Products were run on a 20% non-denaturing acrylamide gel.

### Example 3

### Tma Endo V Active with Bst DNA Polymerase

Tma Endo V, a thermal stable Endo V, was tested for activity in a buffer that may be used for Bst DNA polymerase. Bst DNA polymerase is a thermal stable DNA polymerase. Bst DNA polymerase is active in both the ThermoPol buffer that is provided with the enzyme and NEBuffer 4. Tma Endo V was tested for ability to cleave an inosine containing substrate in each of these buffers as well as a positive control buffer supplied with Tma Endo V from Fermentas. Tma Endo V was active in each of the three buffers, demonstrating that Tma Endo V is active under buffer conditions where Bst DNA polymerase is also active. Products were run on a 20% acrylamide gel.

### Example 4

### Endo V Active with Klenow (Exo Minus)

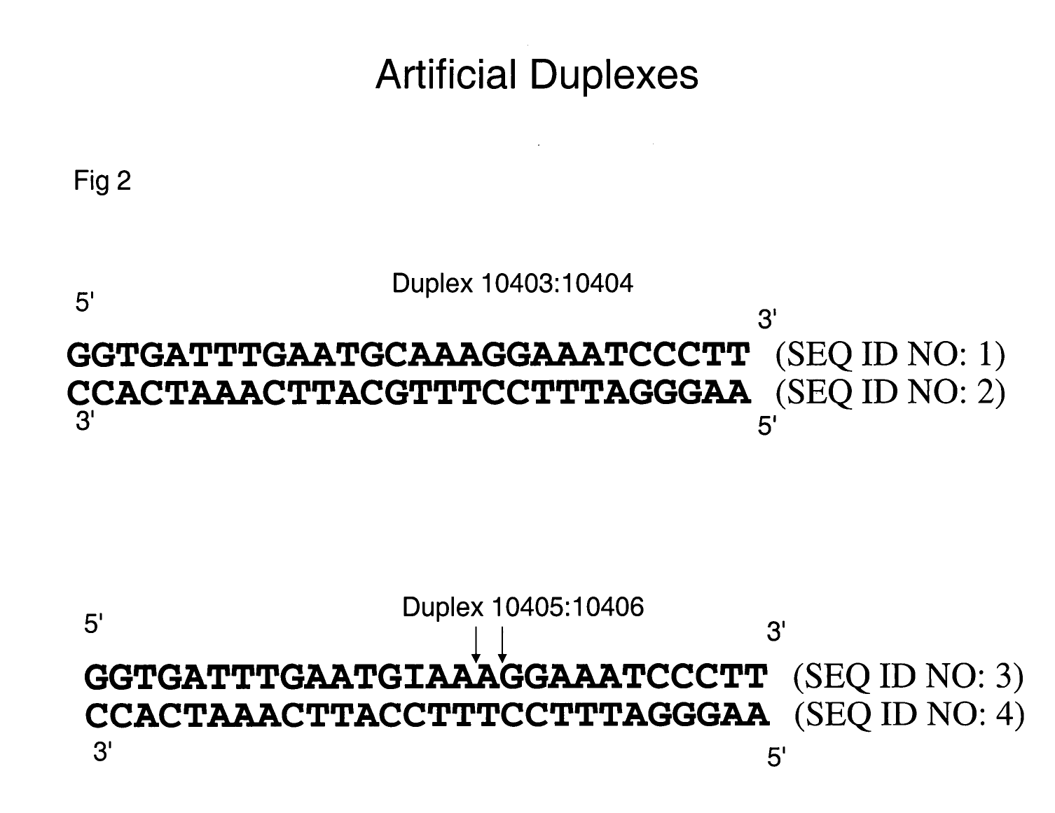

The two duplexes shown in FIG. 2 were incubated with E. coli Endo V alone, Klenow (exo minus) alone, E. coli Endo V plus Klenow or with no enzyme. The products were separated on a 15% acrylamide, 8 M urea gel and stained with Sybr Gold stain. Fragments migrating at about the expected 16 and 11 or 12 nt size expected from cleavage 3′ of the inosine in duplex 10405:10406 were observed in the samples treated with Endo V and fragments were not observed in the samples containing duplex 10403:10404, which does not have an inosine. An increase in the amount of the 11 or 12 nt fragment was observed only when Endo V and Klenow are present and only when the inosine was included in the primer (duplex 10405:10406 in FIG. 2). The results indicate that nicking and strand displacement is occurring in an isothermal manner, is dependent on the presence of inosine and that the 11/12 nt fragment can be amplified in the presence of a strand displacing DNA polymerase. All reactions were done in 1× ThermoPol Buffer (NEB) with dNTPs present.

### Example 5

### Polymerization is Necessary in an Isothermal Reaction

The duplexes from FIG. 2 were incubated with Klenow alone, with E. coli Endonuclease V alone, with Klenow and E. coli Endo V together or with no enzyme either in the presence or absence of dNTPs. Amplification of the 11/12 nt fragment was only observed in the sample with duplex 10405:10406 and only in the reaction that contained E. coli Endo V, Klenow and dNTPs. In the absence of dNTPs no amplification of the 11/12 nt fragment was observed. Products were separated on a 15% acrylamide, 8 M urea gel and stained with Sybr Gold Stain.

### Example 6

### Polymerases have Various Degrees of Effectiveness

Different combinations of polymerase and Endo V were analyzed. Duplex 10405:10406 was incubated with either E. coli EndoV plus Klenow, E.coli Endo V plus Phi29, or Tma Endo V plus Bst DNA Pol. For controls the duplex was also incubated with each enzyme alone or with no enzyme. Products were separated on a 20% acrylamide gel and stained with Sybr Gold Stain. dNTPs were present in all reactions. The increase in yield of the small product is clearly shown in the sample that was treated with E. coli Endo V plus Klenow (exo minus) as the DNA polymerase. Less product than expected was observed in the sample treated with Bst and Tma and very little product was observed in the sample treated with Phi 29 and E. coli Endo V. This may be the result of degradation of the product. Unlike Klenow which is 3′ to 5′ exo minus, Bst and Phi29 both have 3′ to 5′ exo activity and consequently, may be degrading the small product as it is polymerizing.

### Conclusion

It is to be understood that the above description is intended to be illustrative and not restrictive. Many variations of the invention will be apparent to those of skill in the art upon reviewing the above description. The scope of the invention should be determined with reference to the appended claims, along with the full scope of equivalents to which such claims are entitled. All cited references, including patent and non-patent literature, are incorporated herewith by reference in their entireties for all purposes.

What is claimed is:

1. A method for obtaining multiple copies of a template DNA strand comprising:
(a) annealing a primer to said template DNA strand to form a primer-template complex, wherein said primer comprises an inosine base;
(b) extending the 3′ end of said primer in the presence of a DNA polymerase activity to generate a first extended primer that comprises a primer portion and a first copy of the template sequence;
(c) generating a nick in the extended primer using an endonuclease that generates nicks 3′ of said inosine base;
(d) extending the portion of the primer region that is 5′ of the nick from the nick in the presence of the DNA polymerase, thereby displacing the portion of the extended primer that is 3′ of the nick, including the first copy of the template DNA sequence and generating a second extended primer comprising a primer region and a second copy of the template DNA sequence; wherein said second extended primer comprises an inosine base; and
(e) repeating steps (c) and (d) at least once to obtain multiple copies of the template DNA strand.

2. The method of claim 1, wherein said template DNA is genomic DNA.

3. The method of claim 1, wherein said primer is a locus specific primer.

4. The method of claim 1, wherein said nick does not remove said inosine base.

5. The method of claim 1, wherein said nick occurs about 2-3 nucleotides downstream of said 3′ end of said inosine base.

6. The method according to claim 1, wherein said primer is between 15 and 200 bases in length.

7. The method according to claim 1, wherein said primer is between 15 and 100 bases in length.

8. The method according to claim 1, wherein said primer is between 15 and 50 bases in length.

9. The method of claim 1, wherein said DNA polymerase with strand displacement function is selected from the group consisting of Klenow fragment, Bst polymerase, and phi29 polymerase.

10. The method of claim 1, wherein said DNA polymerase is active at a temperature between 30° C. and 80° C.

11. The method according to claim 9, wherein said Bst DNA polymerase is active between 50° C. to 65° C.

12. The method according to claim 9, wherein said phi29 DNA polymerase is active at between 30° C. and 37° C.

13. The method according to claim 1, wherein said endonuclease is an Endo V.

14. The method of claim 13, wherein said Endo V is from E. coli.

15. The method of claim 13, wherein said Endo V is a thermal stable version.

16. The method according to claim 13, wherein said Endo V is active between 30° C. and 60° C.

17. The method according to claim 13, wherein said Endo V is active at a temperature between 30° C. and 37° C.

18. The method of claim 1, wherein prior to step (a) the DNA template strand is denatured.

19. The method of claim 18, wherein the template strand is denatured by heating at about 95° C.

20. The method of claim 1, wherein said annealing step is performed at about 50° C.

21. The method according to claim 1, wherein steps (c) and (d) are performed in same buffer.

22. The method according to claim 21, wherein said buffer comprises 20 mM Tris-acetate, 50 mM potassium acetate, 10 mM magnesium acetate and 1 mM DTT, pH 7.9 at 25° C.

23. The method according to claim 21, wherein said buffer comprises 10 mM Bis-Tris-Propane-HCl, 10 mM magnesium chloride and 1 mM DTT, pH 7.0 at 25° C.

24. The method according to claim 21, wherein said buffer comprises 50 mM sodium chloride, 10 mM Tris-HCl, 10 mM magnesium chloride and 1 mM DTT, pH 7.9 at 25° C.

25. The method according to claim 21, wherein said buffer comprises 100 mM NaCl, 50 mM Tris HCl, 10 mM magnesium chloride and 1 mM DTT, pH 7.9 at 25° C.

26. The method of claim 1, wherein steps (a)-(d) are performed simultaneously in a single reaction.

27. The method of claim 1, wherein steps (a)-(d) are performed under isothermal conditions.

28. The method of claim 1, wherein the step of annealing a primer to said template DNA strand comprises mixing the primer with Rec A protein to obtain a Rec A coated primer and incubating the Rec A coated primer with the template DNA strand in the presence of an ATP analogue.

29. The method of claim 28, wherein said Rec A protein is an E. coli Rec A protein.

30. A method for amplifying a template DNA comprising:
(a) annealing a primer to the template DNA;
(b) extending the primer in the presence of a strand displacing DNA polymerase and deoxyinosine triphosphate to generate a first extension product comprising inosine;
(c) incubating the product of step (b) with an endonuclease V to generate nicks in the first primer extension product at positions 3′ of the incorporated inosine;
(d) extending from the nicks with a strand displacing enzyme to generate second extension products; and
(e) repeating steps (c) and (d) at least once to generate amplified template DNA.

31. The method of claim 30 wherein the ratio of dITP to dGTP is 1:10.

32. The method of claim 30, wherein the ratio of dITP to dGTP is 1:100.

33. The method of claim 30, wherein the ratio of dITP to dGTP is 1:1000.

34. A method for obtaining multiple copies of a template DNA strand comprising:
(a) annealing a primer to said template DNA strand to form a primer-template complex, wherein said primer comprises a uracil base;
(b) extending the 3′ end of said primer in the presence of a DNA polymerase activity to generate a first extended primer that comprises a primer portion and a first copy of the template sequence;
(c) converting the uracil in the extended primer to an abasic site;
(d) generating a nick in the extended primer using an endonuclease that generates a nick 3′ of an abasic site;
(e) extending the portion of the primer region that is 5′ of the nick from the nick in the presence of the DNA polymerase, thereby displacing the portion of the extended primer that is 3′ of the nick, including the first copy of the template DNA sequence and generating a second extended primer comprising a primer region and a second copy of the template DNA sequence; wherein said second extended primer comprises an inosine base; and
(f) repeating steps (d) and (e) at least once to obtain multiple copies of the template DNA strand.

35. The method of claim 34, wherein the uracil is converted to an abasic site by uracil DNA glycosidase.

36. The method of claim 35 wherein steps (d)-(f) are performed under isothermal conditions.

37. The method of claim 35 where the endonuclease is E. coli Endonuclease V and the strand displacing polymerase is phi 29.

38. The method of claim 35 wherein the endonuclease is Tma Endonuclease V and the strand displacing polymerase is Bst DNA polymerase.

39. A method for amplifying a template DNA comprising:
(a) annealing a primer to the template DNA;
(b) extending the primer in the presence of a strand displacing DNA polymerase and deoxyuracil triphosphate to generate a first extension product comprising uracil;
(c) incubating the extension product with uracil DNA glycosidase to convert uracils to abasic sites;
(d) incubating the product of step (c) with an endonuclease to generate a nick in the first primer extension product at the 2 or 3 position 3′ of one or more of said abasic sites;
(e) extending from the nicks with a strand displacing enzyme in the presence of deoxyuracil triphosphate to generate second extension products;
(f) incubating the second extension products with uracil DNA glycosidase to convert uracils to abasic sites and with an endonuclease to generate a nick at the 2 or 3 position 3′ of one or more of said abasic sites; and
(g) repeating steps (e) and (f) at least once to generate amplified template DNA.
